(19) United States
(12) Patent Application Publication
(10) Pub. No.: US 2011/0256212 A1
(43) Pub. Date: Oct. 20, 2011

(54) Use of 8-Quinolinol and its Analogs to Target Cancer Stem Cells

(75) Inventors: Ying Zhang, Ellicott City, MD (US); Jiangbing Zhou, Baltimore, MD (US); Hao Zhang, Ellicott City, MD (US)

(73) Assignee: The Johns Hopkins University, Baltimore, MD (US)

(21) Appl. No.: 13/032,403

(22) Filed: Feb. 22, 2011

Related U.S. Application Data
(62) Division of application No. 12/375,314, filed on Feb. 19, 2010, now abandoned.
(60) Provisional application No. 60/834,071, filed on Jul. 28, 2006.

(51) Int. Cl. A61K 9/127; A61K 31/47; A61K 31/4709; A61K 31/704; A61K 31/475; A61K 31/4745; A61K 33/24; A61K 31/519; A61K 31/7068; A61K 31/5377; A61K 31/517; A61K 31/7048; A61K 31/513; A61K 31/675; A61K 31/52; A61K 31/7076; A61P 35/00; A61P 35/02; A61P 35/04; C12N 5/095

(52) U.S. Cl. 424/450; 435/375; 435/325; 514/311; 514/312; 514/34; 514/283; 424/649; 514/265.1; 514/49; 514/234.5; 514/266.4; 514/27; 514/274; 514/110; 514/263.3; 514/47

(57) ABSTRACT

8-quinolinol (8Q) and derivatives thereof for use in the treatment of proliferative diseases such as cancer, in particular slow metabolizing quiescent cancer stem cells.

This application claims priority to U.S. provisional application No. 60/834,071, filed Jul. 28, 2006, which is hereby incorporated by reference. All references, patents and publications cited herein are hereby incorporated by reference.

The work disclosed herein was supported in part by NIH grants AI44063 and AI49485 from the National Institutes of Health, USA. The Government of the United States of America has certain rights in the invention.

### BACKGROUND OF THE INVENTION

1. Field of the Invention

The invention relates to 8-quinolinol (8Q) and derivatives thereof for use in the treatment of proliferative diseases such as cancer, in particular slow metabolizing quiescent cancer stem cells.

2. Background Information

Cancer stem cells, defined as an initiation subpopulation of tumor cells or a small population of cancer cells that are capable of giving rise to new tumor, were first demonstrated in acute myelogenous leukemia (AML) by John Dick and colleagues¹⁻³. The biology of cancer stem cells is poorly understood and cancer drugs developed to date cannot eliminate cancer stem cells. Although progress has been made on leukemia stem cell research, cancer stem cell research did not draw much attention until the first solid tumor stem cells, breast cancer stem cells, were reported by Clarke and colleagues⁴. These authors isolated a population of highly tumorigenic cells from human breast cancer clinical specimens⁴. This highly tumorigenic subpopulation expressed the CD44⁺CD24^{-/low} surface markers and had the capacity to form tumors following transplantation into NOD/SCID mice. As few as one hundred CD44⁺CD24^{-/low} cells were able to form tumors, whereas tens of thousands of the CD44⁻/CD24^{-/low} cells did not⁴. A variety of cancer stem cells have been since identified in different tumors, including multiple myeloma⁵, human brain tumors⁶⁻⁹, retinoblastoma¹⁰, and melanomas¹¹.

The study of cancer stem cells is important as it could provide new approaches for cancer treatment. Existing therapeutic approaches may eradicate the bulk of a tumor but spare the cancer stem or initiating cells^{2,12-15}. It is conceivable that targeting cancer stem cells will eradicate tumor-initiating cells^{2,12-15}. Because of the quiescence of (cancer) stem cells and their high expression of the ABC (ATP binding cassette) transporter genes which encode efflux pump proteins capable of extruding commonly used drugs, the cancer stem cells have proved difficult to eradicate by current clinical drugs^{14,16,17}. Both leukemia stem cells and glioblastoma stem cells have decreased sensitivity to commonly used clinical drugs^{14,17,18}. Even imatinib (Gleevec), an effective drug for chronic myeloid leukemia (CML), was unable to eradiate CML leukemia stem cells^{19,20}. Thus, there remains an urgent need for new pharmaceutical compounds and compositions to effectively target cancer stem cells.

Human breast cancer is the most common malignancy among women in Western countries^{21,22}. Sphere culture method has been widely used in neural stem cell research and shown to enrich stem cells²³⁻²⁵. In particular, sphere culture methods have been successfully used to isolate brain cancer stem cells^{6,8,26}. Very recently, sphere culture method was also used to culture human breast stem cell or breast cancer stem cells and shown to enrich both normal and cancer stem/progenitor cells²⁷⁻³⁰.

### SUMMARY

The present inventors developed a sphere culture derived from the breast cancer cell line MCF7. The sphere cells were enriched with cancer stem cells expressing surface marker CD44⁺/CD24⁻, exhibiting higher colony forming ability, having increased resistance to cancer drugs and higher expression of ABC transporters than parental cells. The NF-κB pathway was found to be important for the sphere cell survival. Using the sphere cells as a model for cancer stem cells, the inventors screened a compound library and identified a class of compounds having preferential activity for sphere cells.

Accordingly, the present invention provides compounds, pharmaceutical compositions, methods and kits for the treatment of proliferative diseases and disorders such as tumors, in particular the treatment of cancer. Described herein are compounds of formula

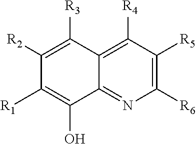

wherein R₁-R₆ independently represent, for example, hydrogen, hydroxyl, a halide, lower alkyl or alkoxy, or a long or short chain fatty acid or ester, pharmaceutically acceptable salts thereof, compositions containing the compounds, and methods of use. These compounds include the compound designated NSC125034 and its analog 8-quinolinol (8Q), and its salt form 8-hydroxyquinol hemisulfate salt, a metal chelator and NF-κB inhibitor.

In one aspect, the invention provides a pharmaceutical composition comprising a pharmaceutically acceptable carrier or excipient and an effective amount of at least one compound of formula:

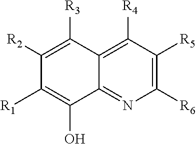

wherein R₁-R₆ independently represent, for example, hydrogen, hydroxyl, a halide, lower alkyl or alkoxy, or a long or short chain fatty acid or ester, and pharmaceutically acceptable salts thereof.
Examples of such compounds are

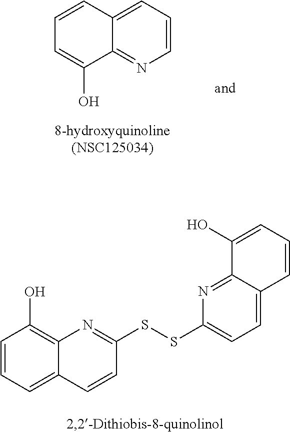

The pharmaceutical composition may optionally further comprise an effective amount of at least one secondary chemotherapeutic agent selected, for example, from the group consisting of paclitaxel, doxyrubicin, vinblastine, and vincristine, Vinorelbine, Topotecan, Carboplatin, Cisplatin, Pemetrexed, Irinotecan, Gemcitabine, Gefitinib, Erlotinib, Etoposide, Fluorouracil, cyclophosphamide, Mercaptopurine, Fludarabine, Ifosfamide, Procarbazine, Mitoxantrone.

The pharmaceutical composition may be formulated for any suitable route of administration, for example, intranasal administration; oral administration; inhalation administration; subcutaneous administration; transdermal administration; intradermal administration; intra-arterial administration, with or without occlusion; intracranial administration; intraventricular administration; intravenous administration; buccal administration; intraperitoneal administration; intraocular administration; intramuscular administration; implantation administration; topical administration; intratumor administration, and central venous administration.

Pharmaceutically acceptable carriers and excipients include any those known in the art, for example, alcohols, dimethyl sulfoxide (DMSO), phosphate buffered saline, saline, a lipid based formulations, liposomal formulations, nanoparticle formulations, micellar formulations, water soluble formulations, and biodegradable polymers.

It is also an object to provide a method of treating cancer in a subject comprising administering to the subject an effective amount of a compound of formula:

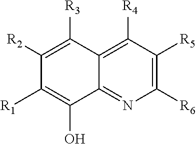

wherein R₁-R₆ independently represent, for example, hydrogen, hydroxyl, a halide, lower alkyl or alkoxy, or a long or short chain fatty acid or ester, or a pharmaceutically acceptable salt thereof, optionally along with a pharmaceutically acceptable carrier or excipient. Examples of compounds to be used in the method are 8-quinolinol and 8-Hydroxyquinol hemisulfate salt. Effective amounts to be administered can be determined by the skilled practitioner without undue experimentation. The appropriate dose to be administered depends on the subject to be treated, such as the general health of the subject, the age of the subject, the state of the disease or condition, the weight of the subject, the size of the tumor, for example. It is expected that dosages of 0.1-100 mg/kg/day, preferably 1-10 mg/kg will be typical. Generally, between about 0.1 mg and about 500 mg or less may be administered to a child and between about 0.1 mg and about 3 grams or less may be administered to an adult.

In an alternative embodiment, dosage is defined in terms of the amount of agent delivered to a target tissue (e.g. a tumor, or an organ). In this instance, dosages may be defined as concentrations, e.g. 0.01 μM-10 mM, 0.01 μM-100 mM, etc.

The methods should be useful for treating a variety of cancers, including solid tumors, lymphomas and leukemias. Examples of tumors for which the treatment methods should be useful include brain tumors, nasal tumors, pharyngeal tumors, head tumors, neck tumors, liver tumors, kidney tumors, prostate tumors, breast tumors, bladder tumors, pancreatic tumors, stomach tumors, colon tumors, ovarian tumors, cervical tumors, and skin tumors; as well as metastases thereof.

Routes of administration include, for example, intranasal administration; oral administration; inhalation administration; subcutaneous administration; transdermal administration; intradermal administration; intra-arterial administration, with or without occlusion; intracranial administration; intraventricular administration; intravenous administration; buccal administration; intraperitoneal administration; intraocular administration; intramuscular administration; implantation administration; topical administration and central venous administration, and intratumor administration.

Pharmaceutically acceptable carriers or excipients for use in the method include, for example, alcohols, dimethyl sulfoxide (DMSO), physiological salines, lipid based formulations, liposomal formulations, nanoparticle formulations, micellar formulations, water soluble formulations, biodegradable polymers, aqueous preparations, hydrophobic preparations, lipid based vehicles, and polymer formulations.

The pharmaceutical composition administered can be in the form of a powder, an aerosol, an aqueous formulation, a liposomal formulation, a nanoparticle formulation, hydrophobic formulation et al.

It has been found that the compounds of the invention exhibit an additive or synergistic effect with other types of chemotherapeutic agents. 8Q and analogs thereof may thus be used as primary chemotherapeutic agents with a variety of cytotoxic agents that are used as chemotherapeutic agents for cancerous or benign tumors, for example, doxyrubicin, vinblastine, paclitaxel, and vincristine, Vinorelbine, Topotecan, Carboplatin, Cisplatin, Pemetrexed, Irinotecan, Gemcitabine, Gefitinib, Erlotinib, Etoposide, Fluorouracil, cyclophosphamide, Mercaptopurine, Fludarabine, Ifosfamide, Procarbazine, Mitoxantrone. As used herein, such additional chemotherapeutic agents will be referred to as “secondary” chemotherapeutic agents. When in combination with 8Q analogs, reduced concentrations of these secondary chemotherapeutic agents should be sufficient to achieve high efficacy. A listing of additional commonly used chemotherapeutic agents to be included in the meaning of “secondary chemotherapeutic agents” as used herein can be found in Blagosklonny, Cell Cycle 3: e52-e59 (2004); other cytotoxic compounds will be known to those of skill in the art. The two (or more) compounds may be administered substantially contemporaneously, or at different times.

The active agent can be administered in a single or, more typically, multiple doses. Preferred dosages for a given agent are readily determinable by those of skill in the art by a variety of means. Other effective dosages can be readily determined by one of ordinary skill in the art through routine trials establishing dose response curves. The amount of agent will, of course, vary depending upon the particular agent used.

The frequency of administration of the active agent, as with the doses, will be determined by the practitioner based on age, weight, disease status, health status and patient responsiveness. Thus, the agents may be administered one or more times daily, weekly, monthly or as appropriate as conventionally determined. The agents may be administered intermittently, such as for a period of days, weeks or months, then not again until some time has passed, such as 3 or 6 months, and then administered again for a period of days, weeks, or months.

Kits with multiple or unit doses of the pharmaceutical compounds and compositions are also included in the present invention. In such kits, in addition to the containers containing the multiple or unit doses of the compositions containing the pharmaceutical compounds and compositions, may also include an informational package insert with instructions describing the use and attendant benefits of the drugs in treating pathological conditions.

In another aspect, the invention provides a method of inhibiting, arresting or killing a cancer stem cell, the method comprising administering an effective amount of a compound of formula:

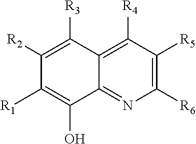

wherein R₁-R₆ independently represent hydrogen, hydroxyl, a halide, lower alkyl or alkoxy, or a long or short chain fatty acid or ester, or pharmaceutically acceptable salts thereof to the cancer stem cell. Compounds mentioned above, including 8-quinolinol, will be useful in the method. Effective dosages can be determined by those of skill in the art and are expected to be in the range of 0.01 μM to about 100 mM, preferably about 0.01 μM to about 10 mM. This method should be useful for the types of cancer mentioned above, in connection with the method for treating cancer, and can be carried out in vivo or in vitro.

In yet another aspect, the invention provides a method of obtaining a purified culture of cancer stem cells comprising the steps of using flow cytometry sorting to obtain side population (SP) enriched in cancer stem cells from a tumor or a cancer cell line, culturing the side population cells to form spheres that are further enriched with cancer stem cells. In particular, a purified culture of stem cells from human breast cancer cell line MCF7 is provided. The invention also provides a purified culture of cancer stem cells derived by the method. The purified culture of cancer stem cells can be used in a method to screen for anticancer agents, wherein test compounds are contacted with the cancer stem cells and their effects on cell growth and/or viability are determined. Thus, the invention provides a method of screening for an anticancer agent comprising contacting a test compound with the purified culture of cancer stem cells, and measuring growth and/or viability, wherein a compound that reduces growth and/or viability is a candidate compound, e.g. for further testing as an anticancer pharmaceutical agent.

### BRIEF DESCRIPTION OF THE DRAWINGS

FIGS. 1A-1E. MCF7 sphere cell morphology and characteristics. A: MCF7 sphere cell morphology. Single sphere cell after 14 days of cultivation was photographed under bright field under inverted microscope. The diameter of the sphere shown in this picture was about 250 μm. B: Surface marker analysis of MCF7 sphere cells. MCF7 cells were stained with Hoechst 33342, then 10⁶ cells were aliquoted and further stained with antibodies including CD44-PE-Cy5 and CD24-PE. The cells were stained with the antibodies on ice for 30 minutes and kept on ice until flow cytometry analysis. C: Comparison of sphere formation ability and colony formation ability of MCF7 and MCF7 sphere cells. Experiments were carried out as described in Materials and Methods. Experiments were performed in triplicate. D: Characteristic Hoechst 33342 dye staining profiles of MCF7 and MCF7 sphere cells. Cells were stained using Hoechst dye 33342 and fluorescence displayed at two wavelength emissions, blue and red. SP region was indicated by trapezoids within each flow diagram. E: Flow cytometry analysis of MCF7 and MCF7 sphere cell cycle status. Single MCF7 and MCF7 spheres were obtained and stained with PI for the quantification of DNA as described in Methods. Distribution of G1/G0 and G2/M phase cells was shown in the figure.

FIG. 2. Susceptibility of MCF7 sphere cells to common anticancer drugs compared with MCF7 cells. Single MCF7 and MCF7 sphere cells were inoculated into 96 well plates at a concentration of 5000 cells per well. After overnight culture, cells were subject to selected clinical drug treatment at the indicated dose for three days. Cell proliferation was determined by MTT assay.

FIGS. 3A-3B. A: Susceptibility of MCF7 and MCF7 sphere cells PI3K pathway inhibitor, rapamycin and NF-κB pathway inhibitors, PTL and PDTC at indicated doses. Experiment was performed as described in FIG. 2 and Materials and Methods. B: Inhibition of NF-κB activity of MCF7 and MCF7 sphere cells after treatment with PTL and PDTC at indicated concentrations. Cells were treated with these compounds at indicated doses for 24 hours. Nuclear extract was prepared from control and treated cells. The quantification of NF-κB activation was performed according to product manual.

FIGS. 4A-4D. Self-renewal ability assays of MCF7 and MCF7 sphere cells. A: Soft agar colony formation assay of MCF7 and MCF7 sphere cells, and these cells with overexpression and knockdown expression of p65. B: Sphere formation assay of MCF7 and MCF7 sphere cells, and these cells with overexpression and knockdown expression of p65. C: Western Blot confirmation of p65 overexpression by transfection of p65 plasmid. D. Western Blot confirmation the expression of p65 in MCF7 sphere cells transfected with dsRNA oligo.

FIGS. 5A-5F. A: Susceptibility of MCF7 sphere cells to compound NSC 125034, compared with MCF7 cells. Experiment was performed as described in FIG. 3 and Materials and Methods. B: Structure of NSC 125034 and 8Q. C: Susceptibility of MCF7 sphere cells to compound 8Q, compared with MCF7 cells. D: In vivo effect of 8Q (4 mg/Kg) by intratumor injection. Nude mouse xenograft assays using MDA-MD-435 human breast cancer cells was carried out using 8Q or paclitaxel or a combination of the two compounds. Mice were treated twice weekly for 3 weeks. Treatment was indicated in the figure as arrows. Tumor sizes were measured weekly. E: In vivo effect of 8Q (20 mg/Kg) by tail vein injection on two different types of breast cancer cell lines, MCF7 (estrogen dependent, xenograft model reflect the early-stage breast malignancy) and MDA-MB435 (estrogen interdependent, xenograft model reflect the later-stage breast malignancy). Mice were treated twice weekly for 4 weeks. (1) MCF7 xenograft (2) MDA-MB435 xenograft. F: Total body weight of individual mouse in the tail vein injection experiment was determined weekly and the average body weight was plotted.

### DETAILED DESCRIPTION

### Definitions

As used herein, the term “lower alkyl” means C₁-C₆ alkyl.

As used herein, the term “lower alkyoxy” means C₁-C₆ alkoxy.

As used herein “halide” refers to fluoride, chloride, bromide, or iodide.

As used herein, “buffers” includes any buffer conventional in the art, such as, for example, Tris, phosphate, imidazole, and bicarbonate.

As used herein, “target tissue” means any tissue to which it is desired to deliver an effective concentration of a compound of the invention, e.g., blood, brain, a specific tumor.

As used herein, the terms “treatment,” “treating,” and the like, refer to obtaining a desired pharmacological and/or physiological effect. The effect may be prophylactic in terms of completely or partially preventing a condition or disease or symptom thereof and/or may be therapeutic in terms of a partial or complete cure for a condition or disease and/or adverse affect attributable to the condition or disease. “Treatment,” thus, for example, covers any treatment of a condition or disease in an animal, particularly in a human, and includes: (a) preventing the condition or disease (e.g. cancer) from occurring in a subject which may be predisposed to the condition or disease but has not yet been diagnosed as having it; (b) inhibiting the condition or disease, such as, arresting its development; and (c) relieving, alleviating or ameliorating the condition or disease, such as, for example, causing regression of the condition or disease.

A “pharmaceutically acceptable carrier” refers to a non-toxic solid, semisolid or liquid filler, diluent, encapsulating material or formulation auxiliary of any conventional type. A “pharmaceutically acceptable carrier” is non-toxic to recipients at the dosages and concentrations employed, and is compatible with other ingredients of the formulation. For example, the carrier for a formulation containing the present compounds preferably does not include oxidizing agents and other compounds that are known to be deleterious to such. Suitable carriers include, but are not limited to, water, dextrose, glycerol, saline, ethanol, buffer, dimethyl sulfoxide, Cremaphor EL, and combinations thereof. The carrier may contain additional agents such as wetting or emulsifying agents, or pH buffering agents. Other materials such as anti-oxidants, humectants, viscosity stabilizers, and similar agents may be added as necessary.

A “pharmaceutically acceptable salt” or “physiologically acceptable salt” refers to a nontoxic salt form of a compound that is suitable for therapeutic administration. Information on suitable physiologically acceptable salts and carriers can be found, inter alia, in Gennaro, A. (1995). “Remington: The Science and Practice of Pharmacy”, 19th edition, Lippincott, Williams, & Wilkins; Ansel, H. C. et al. (1999), Pharmaceutical Dosage Forms and Drug Delivery Systems eds., 7^{th} ed., Lippincott, Williams, & Wilkins; Kibbe, A. H. (2000) Handbook of Pharmaceutical Excipients, eds., 3^{rd} ed. Amer. Pharmaceutical Assoc. Pharmaceutically acceptable salts herein include, inter alia, acid addition salts which are formed with inorganic acids such as, for example, hydrochloric or phosphoric acids, or such organic acids as acetic, mandelic, oxalic, and tartaric. Salts may also be derived from inorganic bases such as, for example, sodium, potassium, ammonium, calcium, or ferric hydroxides, and such organic bases as isopropylamine, trimethylamine, 2-ethylamino ethanol, and histidine.

By “effective amount” or “therapeutically effective amount” is meant an amount that will cause a measurable effect or therapeutic effect, either when delivered as a single dose, or when delivered continuously or repeatedly over a time period (e.g. minutes, hours, days, weeks or months).

The term “pharmaceutically acceptable excipient,” includes vehicles, adjuvants, or diluents or other auxiliary substances, such as those conventional in the art, which are readily available to the public. For example, pharmaceutically acceptable auxiliary substances include pH adjusting and buffering agents, tonicity adjusting agents, stabilizers, wetting agents and the like.

The terms “individual”, “subject,” “host,” and “patient,” are used interchangeably herein to refer to an animal being treated with the present compositions, including, but not limited to, simians, humans, felines, canines, equines, bovines, porcines, ovines, caprines, mammalian farm animals, mammalian sport animals, and mammalian pets.

As used herein “parenteral administration” herein means intravenous, intra-arterial, intramuscular, subcutaneous, transdermal, intradermal and intraperitoneal administration.

A “substantially purified” compound in reference to the compounds described herein is one that is substantially free of compounds that are not the compound in question. By “substantially free” is meant at least 50%, preferably at least 70%, more preferably at least 80%, and even more preferably at least 90%, most preferably greater than 95% or 99% free of extraneous materials.

A “purified cell culture” is one in which greater than 50% of the cells, preferably greater than 75%, 80%, 85%, 90%, 95%, 99% of the cells in the culture are of the specified phenotype. (Cultures described in the examples below have a purity of about 78.5%).

It is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

As used herein, “about” means±10%.

As used herein, the singular forms “a”, “an”, and “the” include plural referents unless the context clearly dictates otherwise. Thus, for example, reference to “a compound” includes a plurality of such compounds and equivalents thereof known to those skilled in the art.

### Methods

MCF7 and MCF7 sphere cell culture. Human breast cancer cell line MCF7 cells, MDA-MB-435 cells were obtained from ATCC (American Type Culture Collection). Cells were grown in Dulbecco's Modified Eagle's Medium (DMEM) (Invitrogen) supplemented with 10% fetal bovine serum (FBS) (Invitrogen), 100 units/ml penicillin and 100 ug/ml streptomycin (Invitrogen), in a 37° C. incubator containing 5% CO₂. To isolate and culture MCF7 sphere cells, SP cells were sorted based on protocol shown below. Sphere cell culture was performed according to published protocol with modifications²⁸⁻³⁰. Briefly, single cells were plated in ultralow attachment plates (Corning, N.Y.) at a density of 20,000 viable cells/mL in primary culture and 1000 cells/mL in passages. Cells were grown in a serum-free mammary epithelial growth medium without bovine pituitary extract (MEGM, BioWhittaker), but supplemented with serum substitute B27 (Invitrogen), 20 ng/mL, Epidermal Growth Factor (EGF), 20 ng/mL, and 20 ng/mL basic fibroblast growth factor (bFGF) (BD Biosciences). In order to passage sphere cells, spheres were collected into 15 ml conic tubes and allowed to settle for 15 minutes. Supernatant was removed. Sphere cells were dissociated enzymatically with 0.05% trypsin, 0.5 mM EDTA (Invitrogen) and mechanically by a glass Pasteur pipette. The cells obtained from dissociation were passed through a 40-μm sieve and analyzed microscopically for single cells and subjected to the experiments below.

Hoechst 33342 staining and flow cytometry analysis/sorting. To identify and sort SP and non-SP fractions, cells were washed with phosphate buffered saline (PBS) and detached from the culture dish with trypsin and EDTA, pelleted by centrifugation, and resuspended in 37° C. DMEM containing 2% FBS at 1×10⁶ cell/ml. Cell staining was performed according to the protocol originally developed by Goodell et al with slight modification⁴⁶. The cells were then incubated with Hoechst 33342 (Sigma) at 5 μg/ml for 90 mM at 37° C. Following staining, the cells were spun down and resuspended in Hanks'Balanced Salt Solution (HBSS) (Invitrogen) containing 1 μg/ml propidium iodide and maintained at 4° C. for flow cytometry analysis/sorting. Cell analysis and sorting were performed on a MoFlo cytometer (Dako Cytomation, Fort Collins, Colo. USA) equipped with a Coherent Enterprise II laser emitting MLUV at 351 nm and blue 488 nm lines. The Hoechst 33342 emission was first split using a 610 dsp filter and then the red and the blue emissions were collected through 670/30 run and 450/65 nm bandpass filters respectively.

Antibody staining. The antibody staining was performed after Hoechst dye staining. After staining with Hoechest 33342, MCF7 cells were spun down and 10⁶ cells were aliquoted, washed and resuspended with washing/staining buffer (PBS supplemented with 1% bovine serum albumin and 0.1% azide). Antibodies used in this study included CD44-PE-Cy5 (eBioscience, San Diego) and CD24-PE (eBioscience, San Diego). Antibody staining was performed on ice for 30 minutes, after which cells were washed with washing/staining buffer for 3 times. Cells stained with antibodies were kept on ice until flow cytometry analysis.

Sphere formation assay. To compare sphere formation ability between MCF7 and MCF7 sphere cells, single cells were seeded at 3000 cells per well in 3 ml of the medium indicated above in 6 well ultralow attachment plates (Corning, N.Y.). To determine the sphere formation ability of MCF7 sphere cells transfected with p65 plasmid and p65 targeted dsRNA oligo, experiments were performed in 24 well ultralow attachment plates (Corning, N.Y.) with inoculation of 1000 cells per well in 1 ml medium. The number of spheres for each well was counted after 7 days of culture. Experiments were done in triplicate.

Soft agar colony formation assay. The colony formation assay was carried out in 35 mm dishes. Briefly, tested cells were plated in 35-mm dishes at 5000 cells/well in 0.35% agar in culture medium over a 0.5% agar layer. Plates were further incubated in cell culture incubator for 12 days until colonies were large enough to be visualized. Colonies were stained with 0.01% Crystal Violet for 1 hour and counted. Experiments were done in triplicate.

Cell proliferation assay. To test the sensitivity of MCF7 and MCF7 sphere cells to specific compounds, both MCF7 and MCF7 sphere single cells were seeded at 3×10⁴ cells/ml in 96-well plates. After overnight incubation, serial concentrations of tested compounds were added. Each concentration was repeated three times. These cells were incubated in a humidified atmosphere with 5% CO₂ for 3 days. Then, 20 ul MTT (3-(4,5-dimethylthiazol-2-yl)-2,5-diphenyl tetrazolium bromide) (Sigma) solution (4.14 mg/ml) was added to each well and incubated at 37° C. for 4 hours. The medium was removed and formazan was dissolved in DMSO and the optical density was measured at 590 nm using a Bio-assay reader (Bio-Rad, USA). The growth inhibition was determined using: Growth inhibition=(control's O.D.−sample's O.D.)/control's OD.

Cell cycle analysis. Cell cycle analysis was performed on single MCF7 and MCF7 sphere cells. Briefly, single MCF7 and MCF7 sphere cells were washed with PBS, fixed with 70% ethanol and stained with propidium iodide (PI) solution (PI solution contains 0.1% Triton X-100, 0.1 mM EDTA, 50 μg/mL RNase A, and 50 μg/mL PI in PBS) for at least 1 hours or until analysis.

Compound library screen for activity against sphere cells. Small-molecule libraries used in this study were from the NCI/NIH. The NO Structural Diversity Set is a library of 1,992 compounds selected from the approximately 140,000-compound NCI drug depository. These compounds were selected based on various criteria including drug-like structure, uniqueness of pharmacophore, and anticancer activity as determined by cell growth inhibition assays against a panel of human tumor cell lines. Detailed data on the selection, structures, and activities of these diversity set compounds can be found on the NCI. Developmental Therapeutics Program web site (http://dtp.nci.nih.gov/index.html).

Since the MCF7 sphere cells grow very slowly and a limited number of cells were available for the screen, we used a more sensitive cell proliferation assay than MTT assay, i.e., a fluorescence-based cell proliferation assay (CyQUANT Cell Proliferation Assay Kit, Molecular Probe) for in vitro compound screening. Briefly, both MCF7 and MCF7 sphere cells were seeded into 96-well culture plates at 5×10² cells per well and incubated in DMEM with 10% FBS, as described above, at 37° C. in an incubator containing 5% CO₂ for 24 h. After a 72 hour incubation, growth of the cells was determined by the fluorescence-based cell proliferation assay. Fluorescence signal was detected by a Bio-assay reader (Bio-Rad, USA) according to the manufacturer's instructions.

Quantitative RT-PCR. Expression levels of 48 ABC (ATP binding cassette) transporters were measured by real-time quantitative RT-PCR using the SuperScript III Platinum SYBR Green One-Step qRT-PCR Kit (Invitrogen) in ABI Prism® 7300 machine (Applied Biosystems, Foster City, Calif.). Specific primers used were according to Szakacs G et al.⁴⁷ with a change in primers for ABCB1. The primers used for ABCB1 were: F: 5-AGCTGCTGTCTGGGCAAAGATACT-3; R: 5AGATCAGCAGGAAAGCAGCACCTA-3. RT-PCR was carried out on 50 ng total RNA, in the presence of 250 nM specific primers. Following reverse transcription (20 min at 55° C.), the PCR reaction consisted of 45 cycles of denaturation (15 s at 95° C.), annealing (30 s at 58° C.), and elongation (30 s at 72° C.). No-template (water) reaction mixture was included as a negative control.

P65 siRNA-mediated knockdown and over-expression. Single MCF7 and MCF7 sphere cells were prepared and cultured in serum free medium as describe above in ultralow attachment 6 well plates on the day of performing gene knockdown and over-expression. For p65 gene knockdown, the Signalsilence p65 siRNA kit from Cell Signaling Technology (Danvers, Mass.) was used. Experiments were performed according to manufacturer's instructions. Briefly, cells were transfected with 50 nM p65 siRNA or control siRNA for gene knockdown. Twenty four hours after transfection, cells were subjected to the soft agar colony formation assay and sphere formation assay as describe above. Cell lysates were prepared 72 hours after transfection with dsRNA oligo for Western Blot analysis of the effect of the gene knockdown.

In order to over-express the p65 gene⁴⁸, FuGENE 6 reagent (Roche Molecular Laboratory, Indianapolis, Ind.) was used according to manufacturer's instructions. Briefly, diluted transfection solution (3 μl FuGENE 6+97 μl serum-free DMEM medium) was incubated for 5 min and mixed with 1 μg of p65 plasmid for 15 min. The FuGENE 6-DNA mixture was then applied to MCF7 and MCF7 sphere cells. Twenty four hours after transfection, cells were subjected to the soft agar colony formation assay and sphere formation assay as described above. Cell lysates were prepared 72 hours after transfection for the Western Blot experiment.

Western blot analysis. Cells were collected, washed with PBS and lysed in RIPA buffer (0.1% SDS, 1% NP-40, 5 mM EDTA, 0.5% Sodium deoxycholate, 150 mM NaCl and 50 mM Tris-HCl) supplemented with protease Inhibitor Tablet (Roche). After centrifugation, cell extracts were collected and the protein concentration determined using the BCA protein assay kit (Pierce, Rockford, Ill.). About 10 μg of protein was run on SDS-polyacrylamide gel and after electrophoresis the proteins were transferred onto nitrocellulose membranes (Bio-Rad). Membranes were probed with primary antibodies against p65 (Cell Signaling Technology) and then secondary antibody alkaline phosphatase-conjugated anti-mouse immunoglobulin G (Jackson Immunoresearch, Inc.). The color reaction was developed with 5-bromo-4-chloro-3-indolyl phosphate and Nitro Blue Tetrazolium solutions (Sigma).

Nuclear extract preparation. Nuclear extracts were prepared according to the protocol recommend by Active Motif Company (Active Motif, Carlsbad, Calif.). Cells were washed and collected in ice-cold PBS/PIB buffer and resuspended in 10 ml of ice-cold hypotonic buffer containing 20 mM HEPES (pH 7.5), 5 mM NaF, 10 μM Na₂MoO₄, 0.1 mM EDTA. PBS/PIB buffer was prepared by adding 0.5 ml of PIB (125 mM NaF, 250 mM β-glycerophosphate, 250 mM p-nitrophenyl phosphate (PNPP) and 25 mM NaV0₃) to 10 ml of 1×PBS prior to use. Following a 15 min incubation on ice, 50 μl of a 10% Nonidet P-40 solution was added and mixed by gentle pipetting. Nuclei were then pelleted by centrifugation at 14,000×g for 30 seconds, washed with the hypotonic buffer and resuspended in 50 μl ice-cold complete lysis buffer (Active Motif, Carlsbad, Calif.). After the nuclear lysates were centrifuged, supernatants were collected for quantification of NF-κB activation. The protein concentrations in the supernatants were determined using the BCA protein assay kit (Pierce, Rockford, Ill.).

Quantification of NF-κB activation. Trans-AM NF-κB assay, an enzyme-linked immunosorbent assay (ELISA)-based method (Trans-AM NF-κB; Active Motif, Carlsbad, Calif.) was used for NF-κB activity quantification according to the manufacturer's instruction. Briefly, cell nuclear extracts were placed in 96-well plates coated with an oligonucleotide containing the NF-κB consensus sequence, and the presence of active NF-κB was detected by using antibodies specific for p50 subunits that are not complexed to IκB and thus able to bind the consensus sequence. A horseradish peroxidase (HRP) conjugated secondary antibody is used to quantify NF-κB binding by conversion of an applied chromogenic substrate.

Antitumor activity in xenograft model. Female athymic (NCR-nu/nu, Tacomic) nude mice were injected with 2×10⁶ MDA-MD-435 tumor cells subcutaneously, and about 3 weeks later, when the tumor volumes reached 100-250 mm, the mice were divided into four groups such that each group had tumors of a similar volume. Stock solutions of paclitaxel alone, 8-Quinolinol (8Q) alone or in combination with paclitaxel were prepared by dissolving the drugs in a vehicle solution (EtOH:cremophor, 50:50 v/v). Vehicle or paclitaxel alone, 8Q alone and 8Q in combination with paclitaxel stock solution were mixed with saline (10:90 v/v). For intratumor injection, the 8Q dose was 4 mg/kg, given twice weekly for three weeks. For tail vein injection, the 8Q dose was 20 mg/kg, given twice weekly for 4 weeks. The paclitaxel dose was 15 mg/kg. Tumor measurements were done twice a week using traceable digital vernier calipers (Fisher). The tumor volumes were determined by measuring the length (l) and the width (w) and calculating the volume (V=lw²/2). The procedures of r animal care and use were in accordance with institutional and NIH guidelines.

Statistical analysis. The growth inhibition effects were compared by Student's t test. P<0.05 was considered significant. One-way ANOVA analysis was performed to determine the statistical significance of treatment related changes in tumor volume in athymic nude mice. Software R package was used for statistical calculation.

### Example 1

### MCF7 Sphere Cell Culture

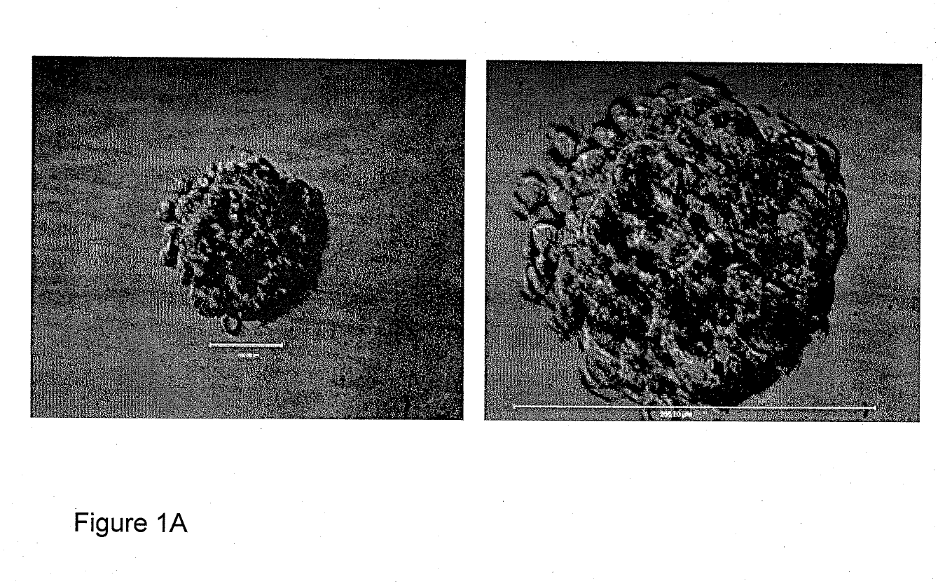

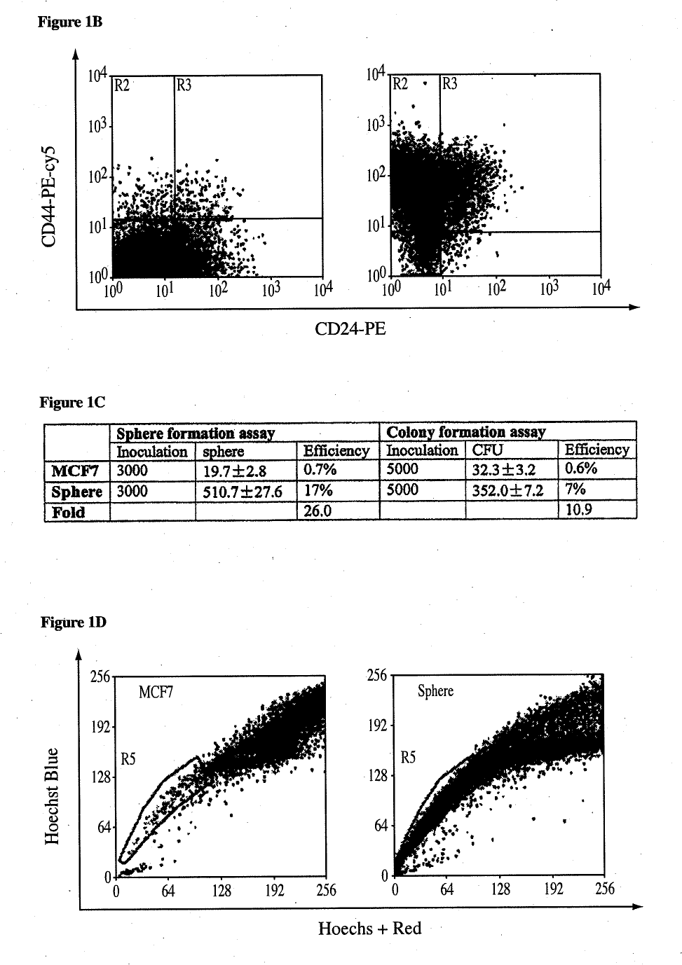

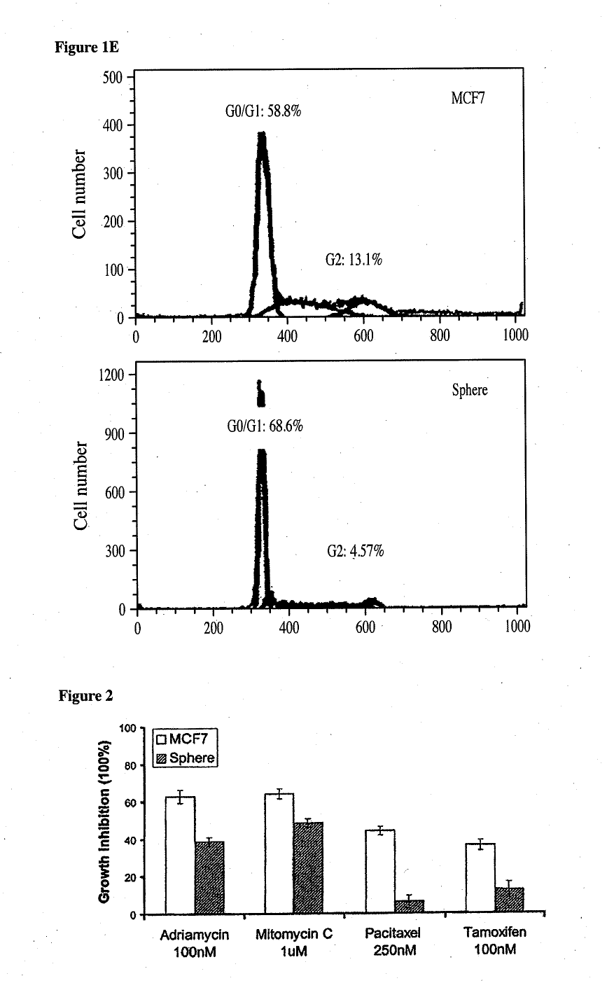

Side population (SP) cells were first defined by Goodell et al in hematopoietic system in 1997 and proved to enrich stem cells³¹. Studies with mammary sphere cells indicate that compared to bulk cells, side population (SP) cells were more capable of forming sphere cells²⁸. In addition, neurosphere cells cultured from SP cells were found to be enriched in stem cells compared to neurosphere cells directly derived from bulk patient samples²⁴. Therefore we attempted to culture MCF7 sphere cells from MCF7 SP cells. The MCF7 SP cells were isolated by flow cytometric cell sorting. The MCF7 sphere cells were then cultured in a serum-free mammary epithelial growth medium, supplemented with B27, 20 ng/mL and EGF and 20 ng/mL bFGF. Using this method we were able to successfully maintain a culture MCF7 sphere cells for more than 1.5 years. The sphere, consisting of many cells associated together, was about 250 μm in diameter (FIG. 1A) and presumably represents many cells associated together from a single clone. As a control, sphere cells cultured from MCF7 non-SP cells could not be passaged for more than 5 generations. The sphere cells were enriched with cancer stem cells expressing surface markers CD44⁺CD24⁻⁴. An increase of 34.7 fold in the breast cancer stem cell population was seen in sphere cells (78.52%) compared with MCF7 parental cells (1.85%) (FIG. 1B). The MCF7 sphere cells did not express lineage-specific markers of mammary epithelium such as CD 10 (myoepithelial lineage marker) and casein (functional alveolar cell marker) (data not shown). Compared to parental MCF7 cells, the sphere cells showed much higher sphere formation ability and soft agar colony formation ability, as shown by 26.0 and 10.9 fold increase, respectively, than the MCF cells (FIG. 1 C). MCF7 sphere cells also had SP properties with high efflux activity. The SP cell fraction in MCF7 sphere cells was 26.81%, compared to 0.87% in parental MCF7 cells (FIG. 1D).

### Example 2

### MCF7 Sphere Cells are Resistant to Common Cancer Drugs

To test the drug sensitivity of the sphere cells, both MCF7 and MCF7 sphere cells were sorted into 96 well plates and treated with various clinical drugs, including adriamycin (doxorubicin), mitomycin C, paclitaxel (Taxol), and tamoxifen, at indicated concentrations for 3 days. Compared to MCF7 cells, MCF7 sphere cells were resistant to all the drugs tested at different concentrations, as shown in FIG. 2. The highest resistance was shown for paclitaxel treatment, which at a concentration of 0.25 uM caused growth inhibition at 44.5% for MCF7 parental cells compared with 6.1% for the MCF7 sphere cells. The drug resistance of MCF7 sphere cells was associated with the relative quiescence of the MCF7 sphere cells. As shown in FIG. 1E, most of MCF7 sphere cells (68.8%) were in G0/GI phase (58.8% for MCF7 cells), and only 4.57% at G2 phase, while the proportion in G2 phase for MCF7 cells was 13.1% (FIG. 1E).

To determine if the high expression of ABC transporters might be responsible for the drug resistance in the sphere cells, we examined the expression levels of all functional 48 ABC transporters in human genome by RT-PCR on MCF7 sphere cells with MCF7 parental cells as a control. Interestingly, some ABC transporters, including ABCA2, ABCB5, ABCCI, ABCC4, ABCC5, ABCC6 and ABCC11, which are known to efflux common drugs and cause drug resistance^{32,33}, were over-expressed by more than 2 fold compared with the control MCF parent cells (Table 1). Sphere cells were also found to over-express several ABC transporters with unknown functions or not known for drug efflux, including ABCA5, ABCA6, ABCA10, ABCA12, ABCB9, ABCB10 and ABCD1. Compared to MCF7 cells, expression of these transporters was 3 fold or higher in the sphere cells. Over-expression of at least some of these ABC transporters may cause the resistance phenotype in the MCF sphere cells.

TABLE 1
Expression of 48 functional ABC transporters of MCF7 sphere cells
compared to MCF7 cells. ABC transporters were quantified by
RT-PCR. Primers were taken from Szakacs G et al⁴⁷. All the ABC
transporters, either up-regulated or down-regulated by more than 2
fold, were shown.

| | Fold change | Function |
|---|---|---|
| ABCA2 | 2.6 ± 0.9 | Efflux of drug Estramustine³³ |
| ABCA4 | 2.0 ± 1.0 | Rod photoreceptor retinoid transport⁴⁹ |
| ABCA5 | 3.1 ± 0.7 | N-retinydilester-PE efflux⁵⁰ |
| ABCA6 | 11.0 ± 3.2 | |
| ABCA7 | 2.8 ± 1.4 | |
| ABCA10 | 4.9 ± 0.8 | |
| ABCA12 | 3.4 ± 1.0 | |
| ABCB5 | 2.1 ± 0.8 | Efflux of drug doxorubicin³² |
| ABCB9 | 3.5 ± 1.4 | |
| ABCB10 | 3.3 ± 1.7 | |
| ABCC1 | 2.5 ± 0.01 | Efflux of drug doxorubicin, daunorubicin, |
| | | vincristine, etoposide, colchicine, |
| | | camptothecins, methotrexate³³ |
| ABCC2 | 0.3 ± 0.4 | Efflux of drug vinblastine, cisplatin, |
| | | doxorubicin, methotrexate³³ |
| ABCC4 | 2.8 ± 1.3 | Efflux of drug 6-Mercaptopurine, |
| | | 6-thioguanine and metabolites, |
| | | methotrexate³³; Nucleoside transport⁴⁸ |
| ABCC5 | 2.1 ± 0.8 | Efflux of drug 6-Mercaptopurine, |
| | | 6-thioguanine and metabolites³³ |
| ABCC6 | 4.0 ± 0.8 | Efflux of drug etoposide³³ |
| ABCC7 | 2.4 ± 2.1 | Chloride ion channel⁴⁹ |
| ABCC11 | 10.3 ± 3.9 | Efflux of drug 5-Fluorouracil³³ |
| ABCC12 | 2.1 ± 1.1 | |
| ABCD1 | 3.0 ± 1.5 | VLCFA transport⁴⁹ |
| ABCD2 | 2.2 ± 0.8 | |
| ABCD3 | 2.1 ± 0.3 | |

### Example 3

### NF-κR Pathway is Important for MCF7 Sphere Cell Survival

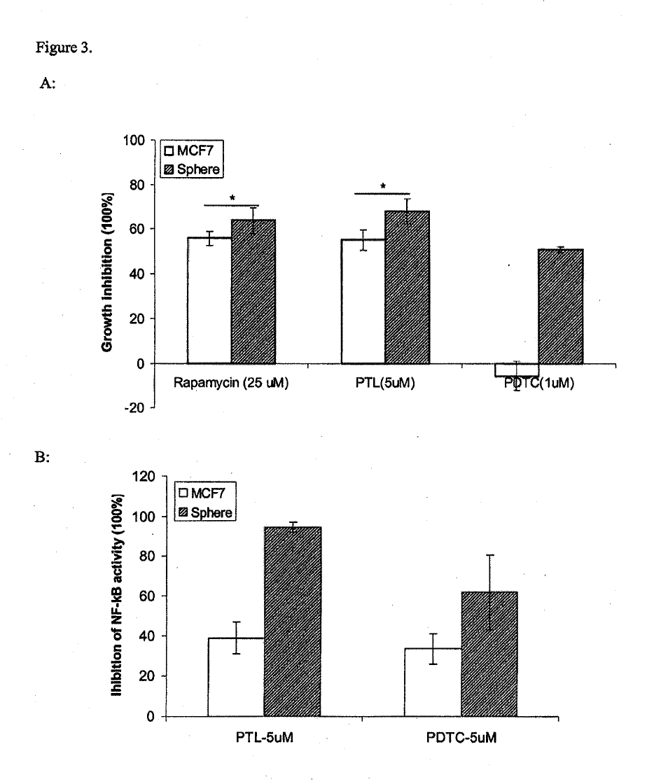

Signaling pathways that show key differences between normal and cancer stem cells could provide therapeutic targets. Wnt, Sonic Hedgehog and Notch pathways were proposed to be active in cancer stem cells and could be important for their self-renewal and survival^{12,21,34,35}. We first determined whether these pathways are important for MCF7 sphere cell survival by comparing the growth inhibition of specific inhibitors of Wnt, Sonic Hedgehog and Notch pathways on MCF7 sphere cells with that for the parental MCF7 cells. However, Wnt pathway inhibitor DKK1, Notch pathway inhibitor GSI1 and Sonic Hedgehog pathway inhibitor cycloparnine showed no higher growth inhibitor ability on MCF7 sphere cells than MCF7 cells (data not shown). Since NF-κB and PI3K pathways are critical for leukemia stem cell survival³⁵⁻³⁸, we tested inhibitors of these pathways for their ability to inhibit MCF7 sphere cells compared with MCF7 cells. Rapamycin, an inhibitor of mTOR, a downstream molecule of PI3K pathway, showed higher growth inhibition effect on MCF7 sphere cells than MCF7 cells (FIG. 3A). Interestingly, NF-κB pathway inhibitors, including parthenolide (PTL) and pyrrolidine dithiocarbamate (PDTC), exerted very marked growth inhibition on MCF7 sphere cells compared to MCF7 cells (FIG. 3A). In particular, PDTC at 1 uM inhibited MCF7 sphere cell growth by 50.8% but had no growth inhibition effects on MCF7 cells. To confirm the NF-κB pathway inhibition effects by these compounds, we quantified the activity of NF-κB by Trans-Am NF-κB assay. As expected, both inhibitors, PTL and PDTC, decreased NF-KB activity (FIG. 3B). For both compounds, the inhibition was greater for MCF7 sphere cells than for MCF7 parental cells. With treatment of 5 μM PTL and PDTC for 24 hours, NF-κB activity in MCF7 sphere cells was decreased by 94.6% and 61.8%, respectively. The same dose of PTL and PDTC only inhibited the NF-κB activity by 38.8% and 33.6%, respectively, in MCF7 cells (FIG. 3B). However, no difference was detected of the NF-κB activity between MCF7 and MCF7 sphere cells (data not shown).

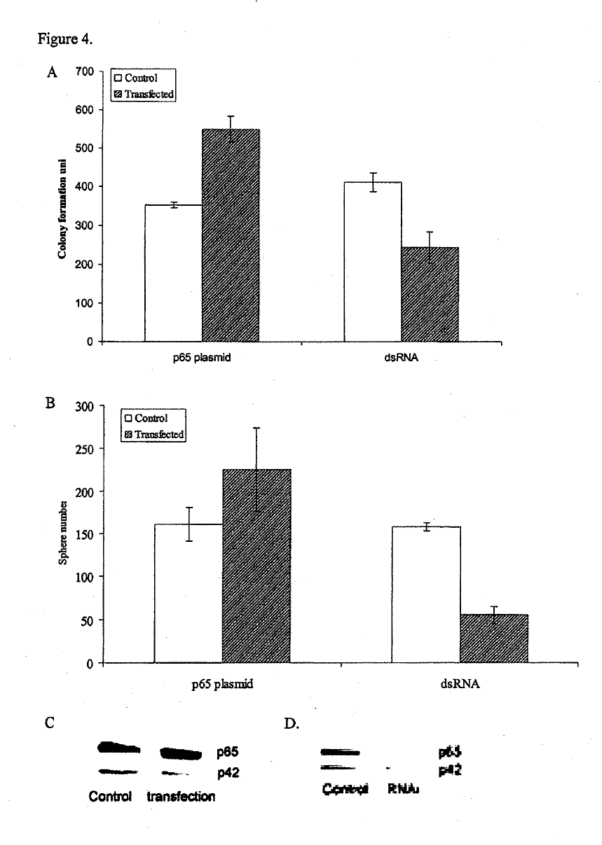

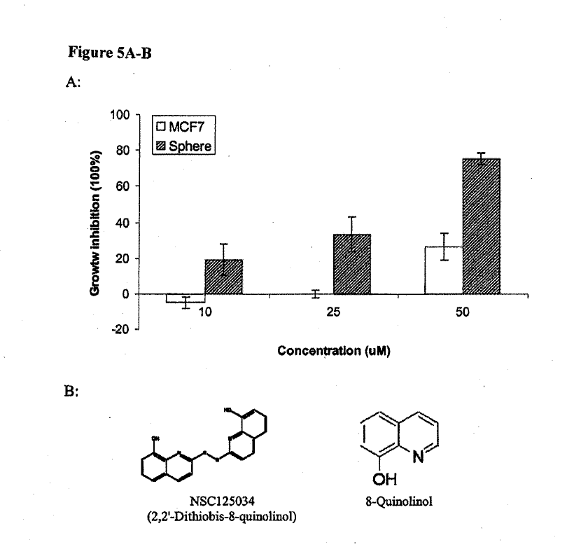

To further confirm the importance of the NF-κB pathway for MCF7 sphere cell self-renewal, we over-expressed and knocked down the expression of the p65 gene, encoding a component of NF-κB by transfection and siRNA. Self-renewal ability of MCF7 sphere cells was determined by soft agar colony formation assay and sphere formation assay. As expected, MCF7 sphere cells over-expressing p65 had higher colony formation ability and sphere formation ability. MCF7 sphere cells over-expressing NF-κB subunit p65 produced 551 colonies and 225 spheres in the same conditions, compared with 325 colonies on soft agar and 161 spheres in their respective controls (FIG. 4 A, B). However, when p65 gene was down-regulated by specific siRNA, MCF7 sphere cells showed decreased ability to form colony on soft agar. As shown in FIGS. 4A and B, MCF7 sphere cells transfected with p65 siRNA formed 244 colonies and 44 spheres, compared with 413 colonies and 158 spheres for the control sphere cells. The over-expression and knockdown of p65 were verified by Western blot (FIG. 4 C, D). A similar effect was observed when another NF-κB subunit, p50, was knocked down by siRNA (data not shown). Taken together, these results suggest that the NF-κB pathway is important for MCF7 sphere cell survival and self-renewal.

### Example 4

### Identification of Compounds that Specifically Inhibit MCF7 Sphere Cells by Compound Library Screening

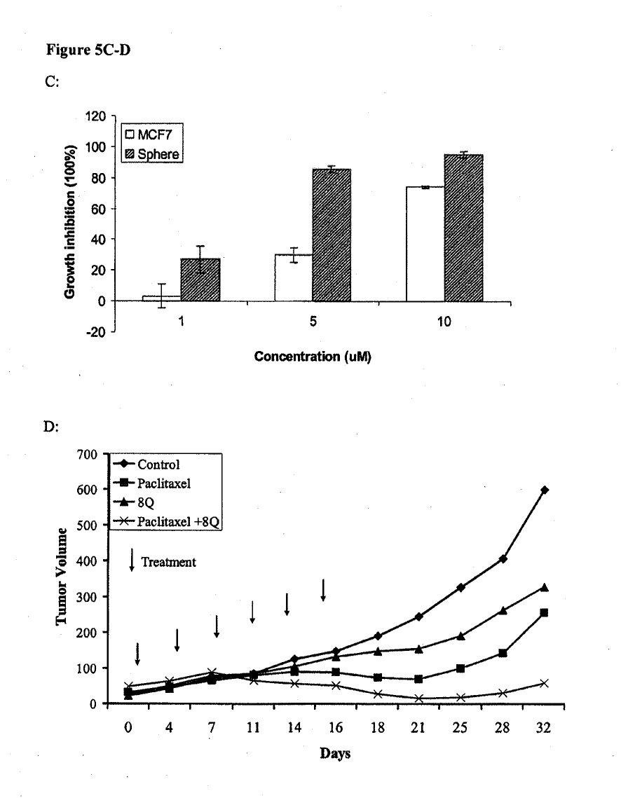

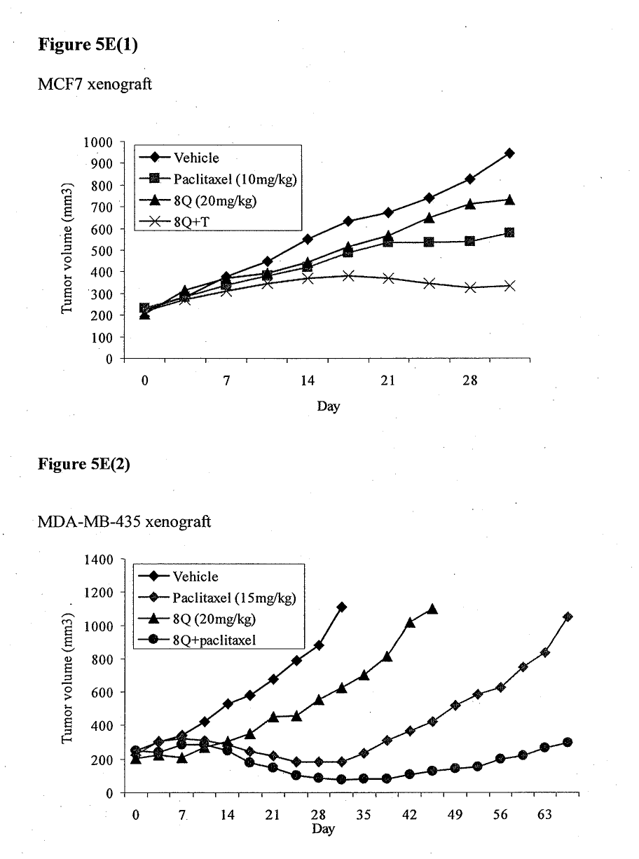

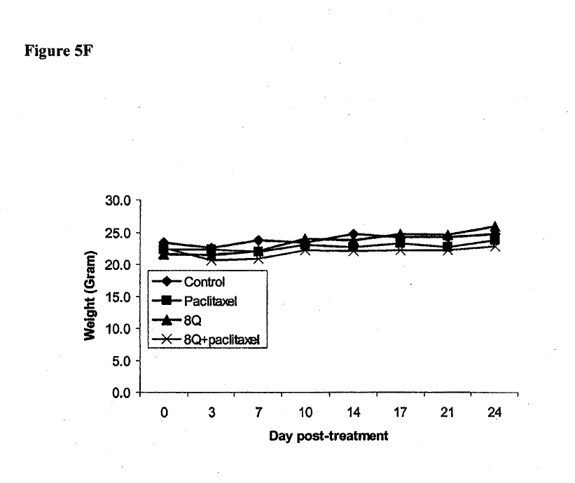

As indicated above, MCF7 sphere cells are resistant to clinical drugs compared with parental MCF7 cells. We sought to identify compounds that preferentially inhibit MCF7 sphere cells over MCF7 cells. For this purpose, we screened the NCI diversity set compound library on both MCF7 and MCF7 sphere cells. One promising compound NSC 125034 was identified to have higher growth inhibition effect on MCF7 sphere cells than MCF7 cells in a dose dependent manner (FIG. 5A). At 25 μM, NSC125034 inhibited sphere cell growth by 33.5%, compared with 0% on MCF7 cells. As NSC125034 has poor solubility even in DMSO, compounds with similar structure were further tested. Ethanol soluble 8Q (8-Quinolinol) was found to have similar or even better activity on MCF7 sphere cells compared with NSC125034 (FIG. 5B, C). For example, at 5 μM, 8Q inhibited sphere cell growth by 86.0% but inhibited MCF7 cells by 30.0% after three day treatment. To further verify the effect of these compounds on potential breast cancer stem cells, we tested these compounds on MCF7 SP cells, another accepted breast cancer stem cell model³⁹⁻⁴¹. Similar to their effects on the sphere cells, 8Q at 5 μM showed better growth inhibition effects on MCF7 SP cells than on MCF7 non-SP cells, with inhibition of 84.7% on SP cells compared to 71.9% on non-SP cells. Since both sphere cells and SP cells are known to the enriched in cancer stem cells³⁹⁻⁴¹, these compounds are thus preferentially active against breast cancer stem cells and were further evaluated for their ability to improve cancer therapy in the mouse model as described below.

### Example 5

### Antitumor Activity of 8Q in the Nude Mouse Tumor Xenograft Model

To demonstrate that the identified compounds can inhibit tumor growth in vivo, we evaluated their antitumor activity in a breast cancer mouse xenograft model. Since MCF7 cell line represents the early stage of breast malignancy and its growth in vivo is dependent on estrogen, we used MDA-MB-435 cell line, which is more malignant, forms tumors more rapidly in mice, and is estrogen independent. Due to poor solubility, NSC 125034 was not used for testing. Compound 8Q was tested in mice bearing MDAMB-435 tumor either alone or in combination with clinical drug paclitaxel. The compound 8Q is a less toxic compound according to U.S. National Toxicology Program acute toxicity studies (http://www.pesticideinfo.org/List_NTPStudies.jsp?Rec_Id=PC34299) and often used as fungicide and microbicide. The LD₅₀s (oral) for mouse, rat and mammal were 20 g/kg, 1.2 g/kg and 1 mg/kg, respectively. 8Q when given through the tail vein at a dose of 20 mg/kg did not show significant toxicity as judged by lack of apparent symptoms and body weight loss (FIG. 5F).

As shown in FIGS. 5D and 5E, the compound 8Q alone significantly inhibited tumor growth in the mouse model either with intratumor injection (4 mg/kg) (P<0.001) or tail vein injection (20 mg/kg) (P<0.001) (FIGS. 5D and 5E). The positive control paclitaxel inhibited the tumor growth as expected. Furthermore, 8Q plus paclitaxel produced an antitumor effect that was superior to paclitaxel or 8Q alone in both intratumor and tail vein injection model. In one of the five mice of the group that received both 8Q and paclitaxel, the tumor completely disappeared. These data suggest a marked synergistic effect on inhibition of tumor growth (FIGS. 5D and 5E(1). We further tested PDTC in another breast cancer xenograft model, MDA-MB-435 xenograft. Since MCF7 represents the early stage of breast malignancy and is easier to be cured, whereas MDA-MB-435 model reflect the late-stage breast malignancy. It will be more valuable if PDTC also shows growth inhibition effect on MDA-MB-435 model. Indeed, PDTC showed similar growth inhibition effect on MDA-MB-435 tumor growth in nude mice either alone or in combination with paclitaxel (FIG. 5E(2)).

As described hereinabove, we successfully established long-term culturable sphere cells from the MCF7 breast cancer cell line. While our work was ongoing, culture and characterization of MCF7 sphere cells was reported by Ponti and colleagues³⁰. The MCF7 sphere cells they identified were enriched for tumorigenic cancer cells with stem/progenitor cell properties³⁰. However, we found that the MCF7 sphere cells cultured by their published protocol could not be passaged more than 5 generations (data not shown). This is presumably because these authors did not use flow cytometry to enrich for SP cells during their sphere cell culture. The MCF7 sphere cells we isolated were enriched in known cancer stem cell surface markers CD44⁺/CD24⁻ and had higher percentage of SP cells with increased efflux activity⁴. The sphere cells had higher colony formation ability (FIG. 1C), and were relatively quiescent and resistant to commonly used cancer drugs. The drug resistance property of MCF7 sphere cells was presumably because of the quiescent state of MCF7 sphere cells as shown by higher percentage of cells in G0/G1 phase and also increased expression of ABC transporters. All these features support the conclusion that the sphere cells we isolated have cancer stem cell properties. Seven ABC transporters known to involve in drug resistance were over-expressed in sphere cells by more than 2 fold (Table 1). In addition, expression of seven other functionally unknown ABC transporters was increased by 3 fold or more in sphere cells. Further studies are needed to confirm the role of these ABC transporters in conferring drug resistance in the sphere cells.

Identifying the pathways that are important for cancer stem cell survival is critical for understanding the biology of cancer stem cells and also for design and development of new drugs that target cancer stem cells. Although Wnt, Sonic Hedgehog and Notch were widely proposed to be active in cancer stem cells and might be important for their self-renewal and survival^{12,21,34,35}, their inhibitors tested in this study, including DKKI, GSI1 and cyclopamine did not inhibit MCF7 sphere cells more than the MCF7 parental cells. Instead, we found that the PI3K pathway and, especially the NF-κB pathway, were critical for the MCF7 sphere cell survival, as demonstrated by their respective pathway inhibitor study. Raparnycin, a specific inhibitor for PI3K pathway, and PTL and PDTC, inhibitors of NF-κB pathway, were shown to specifically inhibit MCF7 sphere cells more strongly than the parental MCF7 cells (FIG. 3A). The importance of NF-κB pathway for MCF7 sphere cell survival was further substantiated by the NF-κB subunit p65 gene over-expression and knockdown experiments (FIG. 4). These findings suggest that PI3K and NF-κB pathways could be good drug targets for breast cancer stem cells.

There is increasing awareness that cancer stem cells pose a significant challenge to effective treatment of cancer as they are resistant to current clinical drugs^{14,17,18,42}. Current cancer drugs were developed by screening and testing on bulk actively growing cancer cells and are not effective for quiescent cancer stem cells. To identify drugs that target cancer stem cells, we screened a compound library using the quiescent and slow growing MCF7 sphere cells as a model for cancer stem cells. One compound, NSC125034, and its analog 8Q were shown to preferentially inhibit the cancer stem cells over bulk cancer cells. The mechanism by which 8Q kills cancer stem cells is not known. However, 8Q as a metal chelator, is believed to bind copper and bring the metal into the cancer cell, thereby causing cytotoxicity⁴³. 8Q may also cause antimitotic effect by inhibiting mitotic kinesin Eg5 ATPase⁴⁴. The most likely possibility is that 8Q may inhibit NF-κB pathway⁴⁵, which has been shown to be important for the sphere cell survival in this study (FIG. 4). Future study is needed to define the mechanism of 8Q inhibition of cancer sphere cells. We next evaluated the antitumor activity of 8Q in vivo in the breast cancer xenograft model. 8Q alone had significant activity in the mouse model but showed a pronounced synergistic effect when combined with clinical drug paclitaxel either by the intratumor injection or tail vein injection (FIGS. 5D and E). Combination of current clinical drugs, which could effectively eliminate bulk cancer cells, and cancer stem cell targeting drugs, like PI3K and NF-κB pathway inhibitors, which could effectively eliminate cancer stem cells, may provide better therapeutic efficacy. Compounds like 8Q, which we have shown to kill a population of quiescent cancer stem cells and to synergize with the other clinical cancer drugs such as paclitaxel, hold promise for improved cancer treatment in the clinic due to the ability to eliminate the root of cancer and achieve longer term remission.

### REFERENCES

References cited herein are listed below for convenience.
1. Bonnet, D. & Dick, J. E. Human acute myeloid leukemia is organized as a hierarchy that originates from a primitive hematopoietic cell. Nat Med 3, 730-7 (1997).
2. Behbod, F. & Rosen, J. M. Will cancer stem cells provide new therapeutic targets? Carcinogenesis 26, 703-11 (2004).
3. Lapidot, T. et al. A cell initiating human acute myeloid leukaemia after transplantation into SCID mice. Nature 367, 645-8 (1994).
4. Al-Hajj, M., Wicha, M. S., Benito-Hernandez, A., Morrison, S. J. & Clarke, M. F. Prospective identification of tumorigenic breast cancer cells. Proc Natl Acad Sci USA 100, 3983-8 (2003).
5. Matsui, W. et al. Characterization of clonogenic multiple myeloma cells. Blood 103, 2332-6 (2004).
6. Singh, S. K. et al. Identification of a cancer stem cell in human brain tumors. Cancer Res 63, 5821-8 (2003).
7. Singh, S. K. et al. Identification of human brain tumour initiating cells. Nature 432, 396-401 (2004).
8. Hemmati, H. D. et al. Cancerous stem cells can arise from pediatric brain tumors. Proc Natl Acad Sci USA 100, 15178-83 (2003).
9. Galli, R. et al. Isolation and characterization of tumongemc, stem-like neural precursors from human glioblastoma. Cancer Res 64, 7011-21 (2004).
10. Seigel, G. M., Campbell, L. M., Narayan, M. & Gonzalez-Fernandez, F. Cancer stem cell characteristics in retinoblastoma. Mol Vis 11, 729-37 (2005).
11. Fang, D. et al. A tumorigenic subpopulation with stem cell properties in melanomas. Cancer Res 65, 9328-37 (2005).
12. Reya, T., Morrison, S. J., Clarke, M. F. & Weissman, I. L. Stem cells, cancer, and cancer stem cells. Nature 414, 105-11 (2001).
13. Al-Hajj, M., Becker, M. W., Wicha, M., Weissman, I. & Clarke, M. F. Therapeutic implications of cancer stem cells. Curr Opin Genet Dev 14, 43-7 (2004).
14. Jones, R. J., Matsui, W. H. & Smith, B. D. Cancer stem cells: are we missing the target? J Natl Cancer Inst 96, 583-5 (2004).
15. Dean, M., Fojo, T. & Bates, S. Tumour stem cells and drug resistance. Nat Rev Cancer 5, 275-84 (2005).
16. Donnenberg, V. S. & Donnenberg, A. D. Multiple drug resistance in cancer revisited: the cancer stem cell hypothesis. J Clin Pharmacol 45, 872-7 (2005).
17. Guzman, M. L. & Jordan, C. T. Considerations for targeting malignant stem cells in leukemia. Cancer Control 11, 97-104 (2004).
18. Costello, R. T. et al. Human acute myeloid leukemia CD34+/CD38− progenitor cells have decreased sensitivity to chemotherapy and Fas-induced apoptosis, reduced immunogenicity, and impaired dendntic cell transformation capacities. Cancer Res 60, 4403-11 (2000).
19. Graham, S. M. et al. Primitive, quiescent, Philadelphia-positive stem cells from patients with chronic myeloid leukemia are insensitive to STI571 in vitro. Blood 99, 319-25 (2002).
20. Angstreich, G. R. et al. Effects of imatinib and interferon on primitive chronic myeloid leukaemia progenitors. Br J Haematol 130, 373-81 (2005).
21. Zhang, M. & Rosen, J. M. Stem cells in the etiology and treatment of cancer. Curr Opin Genet Dev 16, 60-4 (2005).
22. Edwards, B. K. et al. Annual report to the nation on the status of cancer, 19752002, featuring population-based trends in cancer treatment. J Natl Cancer Inst 97, 1407-27 (2005).
23. Suslov, O. N., Kukekov, V. G., Ignatova, T. N. & Steindler, D. A. Neural stem cell heterogeneity demonstrated by molecular phenotyping of clonal neurospheres. Proc Natl Acad Sci USA 99, 14506-11 (2002).
24. Kim, M. & Morshead, C. M. Distinct populations of forebrain neural stem and progenitor cells can be isolated using side-population analysis. J Neurosci 23, 10703-9 (2003).
25. Okano, H. Neural stem cells: progression of basic research and perspective for clinical application. Keio J Med 51, 115-28 (2002).
26. Singh, S. K., Clarke, I. D., Hide, T. & Dirks, P. B. Cancer stem cells in nervous system tumors. Oncogene 23, 7267-73 (2004).
27. Dontu, G. et al. Role of Notch signaling in cell-fate determination of human mammary stem/progenitor cells. Breast Cancer Res 6, R605-15 (2004).
28. Dontu, G. et al. In vitro propagation and transcriptional profiling of human mammary stem/progenitor cells. Genes Dev 17, 1253-70 (2003).
29. Youn, B. S. et al. Large-scale expansion of mammary epithelial stem cell aggregates in suspension bioreactors. Biotechnol Prog 21, 984-93 (2005).
30. Ponti, D. et al. Isolation and in vitro propagation of tumorigenic breast cancer cells with stem/progenitor cell properties. Cancer Res 65, 5506-11 (2005).
31. Goodell, M. A. et al. Dye efflux studies suggest that hematopoietic stem cells expressing low or undetectable levels of CD34 antigen exist in multiple species. Nat Med 3, 1337-45 (1997).
32. Frank, N. Y. et al. ABCB5-mediated doxorubicin transport and chemoresistance in human malignant melanoma. Cancer Res 65, 4320-33 (2005).
33. Schatzlein, A. G. Delivering cancer stem cell therapies—A role for nanomedicines? Eur J Cancer 42, 1309-15 (2006).
34. Guo, W., Lasky, J. L., 3rd & Wu, H. Cancer Stem Cells. Pediatr Res (2006). 35. Jordan, C. T. Cancer stem cell biology: from leukemia to solid tumors. Curr Opin Cell Biol 16, 708-12 (2004).
36. Guzman, M. L. et al. Nuclear factor-kappaB is constitutively activated in primitive human acute myelogenous leukemia cells. Blood 98, 2301-7 (2001).
37. Xu, Q., Simpson, S. E., Scialla, T. J., Bagg, A. & Carroll, M. Survival of acute myeloid leukemia cells requires P13 kinase activation. Blood 102, 972-80 (2003).
38. Rossi, D. J. & Weissman, I. L. Pten, tumorigenesis, and stem cell self-renewal. Cell 125, 229-31 (2006).
39. Kondo, T., Setoguchi, T. & Taga, T. Persistence of a small subpopulation of cancer stem-like cells in the C6 glioma cell line. Proc Natl Acad Sci USA 101, 781-6 (2004).
40. Hirschmann-Jax, C. et al. A distinct “side population” of cells with high drug efflux capacity in human tumor cells. Proc Natl Acad Sci USA 101, 14228-33 (2004).
41. Patrawala, L. et al. Side Population Is Enriched in Tumorigenic, Stem-Like Cancer Cells, whereas ABCG2+ and ABCG2− Cancer Cells Are Similarly Tumorigenic. Cancer Res 65, 6207-19 (2005).
42. Eramo, A. et al. Chemotherapy resistance of glioblastoma stem cells. Cell Death Differ 13, 1238-41 (2006).
43. Daniel, K. G. et al. Clioquinol and pyrrolidine dithiocarbamate complex with copper to form proteasome inhibitors and apoptosis inducers in human breast cancer cells. Breast Cancer Res 7, R897-908 (2005).
44. DeBonis, S. et al. In vitro screening for inhibitors of the human mitotic kinesin Eg5 with antimitotic and antitumor activities. Mol Cancer Ther 3, 1079-90 (2004).
45. Kim, Y. H. et al. 8-Hydroxyquinoline inhibits iNOS expression and nitric oxide production by down-regulating LPS-induced activity of NF-kappaB and C/EBPbeta in Raw 264.7 cells. Biochem Biophys Res Commun 329, 591-7 (2005).
46. Goodell, M. A., Brose, K., Paradis, G., Conner, A. S. & Mulligan, R. C. Isolation and functional properties of murine hematopoietic stem cells that are replicating in vivo. J Exp Med 183, 1797-806 (1996).
47. Szakacs, G. et al. Predicting drug sensitivity and resistance: profiling ABC transporter genes in cancer cells. Cancer Cell 6, 129-37 (2004).
48. Agrawal, A., Cha-Molstad, H., Samols, D. & Kushner, I. Transactivation of Creactive protein by IL-6 requires synergistic interaction of CCAAT/enhancer binding protein beta (C/EBP beta) and Rel p50. J Immunol 166, 2378-84 (2001).
49. Dean, M. & Allikmets, R. Complete characterization of the human ABC gene family. J Bioenerg Biomembr 33, 475-9 (2001).
50. Glavinas, H., Krajcsi, P., Cserepes, J. & Sarkadi, B. The role of ABC transporters in drug resistance, metabolism and toxicity. Curr Drug Deliv 1, 27-42 (2004).

1-6. (canceled)

7. A method of treating cancer in a subject comprising administering to the subject an effective amount of a compound of formula: 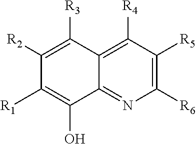
wherein Rᵢ-R₆ independently represent hydrogen, hydroxyl, a halide, lower alkyl or alkoxy, a long or short chain fatty acid or ester, or a pharmaceutically acceptable salt thereof, optionally along with a pharmaceutically acceptable carrier or excipient.

8. The method of claim 7 wherein the compound is 8-quinolinol, 8-hydroxyquinol hemisulfate salt, 2,2′-Dithiobis-8-quinolinol, or a pharmaceutically acceptable salt thereof.

9. The method of claim 7 wherein the dosage is between 1-100 mg/kg/day.

10. The method of claim 8 wherein the dosage is between 5-50 mg/kg/day.

11. The method of claim 7 wherein the cancer is a solid tumor, a lymphoma or a leukemia.

12. The method of claim 11, wherein the cancer is selected from the group consisting of a brain tumor, nasal tumor, pharyngeal tumor, head tumor, neck tumor, liver tumor, kidney tumor, prostate tumor, breast tumor, bladder tumor, pancreatic tumor, stomach tumor, colon tumor, ovarian tumor, cervical tumor, and skin tumor; and metastases thereof.

13. The method of claim 7, wherein the route of administration is selected from the group consisting of intranasal administration; oral administration; inhalation administration; subcutaneous administration; transdermal administration; intradermal administration; intra-arterial administration, with or without occlusion; intracranial administration; intraventricular administration; intravenous administration; buccal administration; intraperitoneal administration; intraocular administration; intramuscular administration; implantation administration; topical administration, intratumor administration and central venous administration.

14. The method of claim 13, wherein the pharmaceutically acceptable carrier or excipient comprises a composition selected from the group consisting of an alcohol, dimethyl sulfoxide (DMSO), a physiological saline, a lipid based formulation, a liposomal formulation, a nanoparticle formulation, a micellar formulation, a water soluble formulation, a biodegradable polymer, an aqueous preparation, a hydrophobic preparation, a lipid based vehicle, and a polymer formulation.

15. The method of claim 7, wherein the composition is in a form selected from the group consisting of a powder, an aerosol, an aqueous formulation, a liposomal formulation, a nanoparticle formulation, and a hydrophobic formulation.

16. The method of claim 7, wherein the method additionally comprises administering an effective amount of a secondary chemotherapeutic agent selected from the group consisting of paclitaxel, doxyrubicin, vinblastine, vincristine, Vinorelbine, Topotecan, Carboplatin, Cisplatin, Pemetrexed, Irinotecan, Gemcitabine, Gefitinib, Erlotinib, Etoposide, Fluorouracil, cyclophosphamide, Mercaptopurine, Fludarabine, Ifosfamide, Procarbazine, Mitoxantrone.

17. The method of claim 16, wherein the two compounds are administered substantially contemporaneously.

18. The method of claim 16, wherein the two compounds are administered at different times.

19. The method of claim 7, wherein the compound(s) is(are) administered intravenously.

20. The method of claim 7, wherein the dosage administered results in a concentration in a target tissue of the subject selected from the group consisting of 0.01 p.M to about 10 mM.

21. The method of claim 7 wherein the cancer is a metastatic cancer.

22-23. (canceled)

24. A method of inhibiting, arresting or killing a cancer stem cell, the method comprising administering an effective amount of a compound of formula: 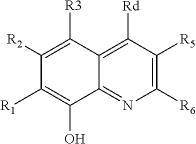
wherein Rᵢ-R₆ independently represent hydrogen, hydroxyl, a halide, lower alkyl or alkoxy, a long or short chain fatty acid or ester, or a pharmaceutically acceptable salt thereof, to the cancer stem cell.

25. The method of claim 24 wherein the compound is 8-quinolinol, 2,2′-Dithiobis-8-quinolinol, or a pharmaceutically acceptable salt thereof.

26. The method of claim 24 wherein the dosage is between 1-200 mg/kg/day.

27. The method of claim 26 wherein the dosage is between 2-100 mg/kg/day.

28-32. (canceled)

33. A method of obtaining a purified culture of cancer stem cells comprising the steps of using flow cytometry sorting to obtain side population (SP) enriched in cancer stem cells; and culturing SP cells under suitable conditions to allow for formation of spheres to obtain the purified culture of cancer stem cells.

34-39. (canceled)
